(19) United States
(12) Patent Application Publication
(10) Pub. No.: US 2025/0288326 A1
(43) Pub. Date: Sep. 18, 2025

(54) THREADED BONE IMPLANT AND SYSTEMS

(71) Applicant: SI-Bone Inc., Santa Clara, CA (US)

(72) Inventors: Francois FOLLINI, Austin, TX (US); Bret SCHNEIDER, San Jose, CA (US); Derek LINDSEY, San Jose, CA (US); Scott YERBY, Montara, CA (US); Jeffrey MULLIN, Buffalo, NY (US); Han Jo KIM, New York, NY (US); Roland KENT, Coeur d'Alene, ID (US); Gregory MUNDIS, San Diego, CA (US); Benjamin ELDER, Rochester, MN (US); Brian O'SHAUGHNESSY, Nashville, TN (US); Avery BUCHHOLZ, Orlando, FL (US); Jay TURNER, Phoenix, AZ (US); Zachary TEMPEL, Cincinnati, OH (US); Frances NGUYEN-KHOA, New York, NY (US); Ian BAILEY, San Jose, CA (US); Nikolas KERR, Milpitas, CA (US)

(21) Appl. No.: 19/078,206

(22) Filed: Mar. 12, 2025

Related U.S. Application Data
(60) Provisional application No. 63/564,291, filed on Mar. 12, 2024.

(51) Int. Cl. A61B 17/70; A61B 17/86

(52) U.S. Cl. CPC A61B 17/7032; A61B 17/863; A61B 2017/8655

(57) ABSTRACT

Threaded bone implants, such as pedicle screws, are provided with an elongate body comprising a first portion, a second portion distal to the first portion, and a third portion distal to the second portion. The first, second and third portions may be provided with different features that correspond to first, second and third anatomical regions of interest in a vertebrae or other bone segment(s). The bone implants, systems, methods of manufacture, and methods of use disclosed herein may be used for the stabilization, fixation and/or fusion of the sacroiliac joint, the spine and or other bone segments.

### CLAIM OF PRIORITY

This application claims the benefit of U.S. Provisional Application No. 63/564,291, filed Mar. 12, 2024, which is herein incorporated by reference in its entirety for all purposes.

### INCORPORATION BY REFERENCE

All references mentioned herein are fully incorporated by reference herein in their entireties.

### FIELD

The present invention generally relates to bone implants. More specifically, the present invention relates to bone implants, systems and methods that may be used for the stabilization, fixation and/or fusion of the sacroiliac joint, the spine and or other bone segments.

### BACKGROUND

Bone implants may be used to assist in the fixation of bones in spine surgeries such as, but not limited to, spinal fusions. Distal regions of bone implants may be configured for threading into a bone, such as a pedicle of a vertebrae. Proximal regions of the bone implants may be configured to be coupled to a “tulip”. The tulip is generally rotatable relative to the implant in a first moveable state, the tulip being further configured to accept a rod for coupling to additional bone implants located in other bone segments.

Existing bone implants are susceptible to undesirable movement after they have been implanted in bone, such as toggle (movement caused by a force perpendicular to the screw long axis) and pullout (movement caused by a force parallel to the screw long axis).

What is needed and not provided by the prior art are bone implants that provide more stability against movement when implanted in bone. These and other advantages are provided by the methods, devices and systems disclosed herein.

### SUMMARY OF THE DISCLOSURE

The patent or application file contains at least one drawing executed in color. Copies of this patent or patent application publication with color drawing(s) will be provided by the Office upon request and payment of the necessary fee.

One aspect of the disclosure is related to threaded bone implants. In some implementations, they may be used as pedicle screws implanted through the pedicle and within the vertebral body, and may be part of a spinal construct system for providing stabilization to one or more portions of a spine. The threaded bone implants herein may be referred to herein generally as screws. Spinal constructs herein (e.g., a combination of screws and rods) may be referred to herein generally as systems. The screws may be implanted in the thoracic spines, and they may additionally or alternatively find applications in the lumbar spine and sacrum.

The screws and/or spinal construct systems herein may provide enhanced stability by promoting bony ingrowth and reducing the likelihood of toggle (movement caused by a force perpendicular to the screw long axis) and pullout following positioning in bone. One or more screw features may be designed based on vertebra morphology and/or the method of implantation to promote screw stability.

### BRIEF DESCRIPTION OF THE DRAWINGS

The novel features of the disclosure are set forth with particularity in the claims that follow. A better understanding of the features and advantages of the present disclosure will be obtained by reference to the following detailed description that sets forth illustrative embodiments, in which the principles of the disclosure are utilized, and the accompanying drawings of which:

FIGS. 1A and 1B illustrate exemplary implant corridors and generally trajectories for implantation through a pedicle and into a vertebral body.

FIG. 2 illustrates an exemplary screw (top screw) according to one embodiment of the present disclosure that includes one or more screw features for added stability as a pedicle screw, compared with an exemplary prior art screw (bottom screw).

FIGS. 3A and 3B illustrate additional exemplary embodiments according to aspects of the present disclosure of screws with common features for increased stability when used as pedicle screws.

FIG. 4 illustrates another exemplary screw coupled at its proximal end to an exemplary tulip.

FIG. 5A represents an exemplary screw herein being rotated into bone during an implantation procedure.

FIG. 5B represents the screw of FIG. 5A fully seated in place with a distal end in the vertebral body.

FIGS. 6A and 6B show side views of an exemplary screw configured for bone harvesting.

FIG. 7A is a side view showing exemplary details of screws herein with bone harvesting features.

FIG. 7B is an enlarged fragmentary view showing exemplary details of screws herein with bone harvesting features.

FIG. 8 is a side view of a screw implanted in bone and showing exemplary details of screws herein with bone harvesting features.

FIGS. 9A and 9B are top views of a portion of a vertebrae and illustrate parts of an exemplary pedicle screw implantation procedure, with FIG. 9A showing a screw partially implanted and FIG. 9B showing it fully seated.

FIG. 10 illustrates an exemplary distal region of any of the screws herein, showing fenestration volume decreasing from distal to proximal direction.

FIG. 11 illustrates an exemplary screw distal region including a self-drilling or self-tapping tip with one or more self-drilling features.

FIG. 12 illustrates an exemplary screw with a plurality of self-filling pores and a self-drilling or self-trapping tip with one or more self-drilling features.

FIGS. 13A and 13B illustrate a distal region of an exemplary screw herein.

FIG. 14 illustrates a distal region of a prior art screw.

FIG. 15 illustrates an exemplary screw implanted through a pedicle.

FIGS. 16A and 16B illustrate side views of an exemplary screw, with lattice sections omitted for clarity in FIG. 16B.

FIG. 17A illustrates the screw of FIGS. 16A and 16B implanted in a pedicle.

FIG. 17B illustrates three anatomical regions of interest in a vertebrae.

FIG. 17C is a bar graph that illustrates relative densities of the three regions of FIG. 17B.

FIGS. 18A and 18B illustrate an exemplary screw adapted for increased stability, illustrating an exemplary dual-single-dual thread arrangement.

FIGS. 19A and 19B illustrate an exemplary screw and show the greater crest thickness of thread 180 compared to secondary lead 182.

FIGS. 20A-20C illustrate an exemplary screw that includes a proximal dual lead section wherein the different threads have different thread heights.

FIGS. 21A, 21B and 21C illustrate an exemplary screw adapted to increase stability.

FIG. 22 illustrates some exemplary deficiencies of prior art machined screws in the event of repositioning in bone.

FIGS. 23A, 23B and 23C illustrate exemplary screw features that can overcome or minimize the deficiencies of the machined screws shown in FIG. 22.

FIGS. 24A, 24B, and 24C illustrate an exemplary feature wherein one or more threads may be toothed.

FIGS. 25A-25E, illustrate an exemplary screw that includes a plurality of cutting teeth on the thread lead at a distal end.

FIGS. 26, 27 and 28 illustrate exemplary core configurations, any of which may be incorporated into any of the screws herein.

FIG. 29 provides two cross-sectional views of screw threads and illustrates an additive manufacturing techniques that can increase stability in screws.

FIG. 30 provides a cross-sectional view of screw threads and illustrates an additive manufacturing techniques that may improve one or more aspects of any of the screws herein or other threaded implants.

FIG. 31 illustrates a midsection of an exemplary screw.

### DETAILED DESCRIPTION

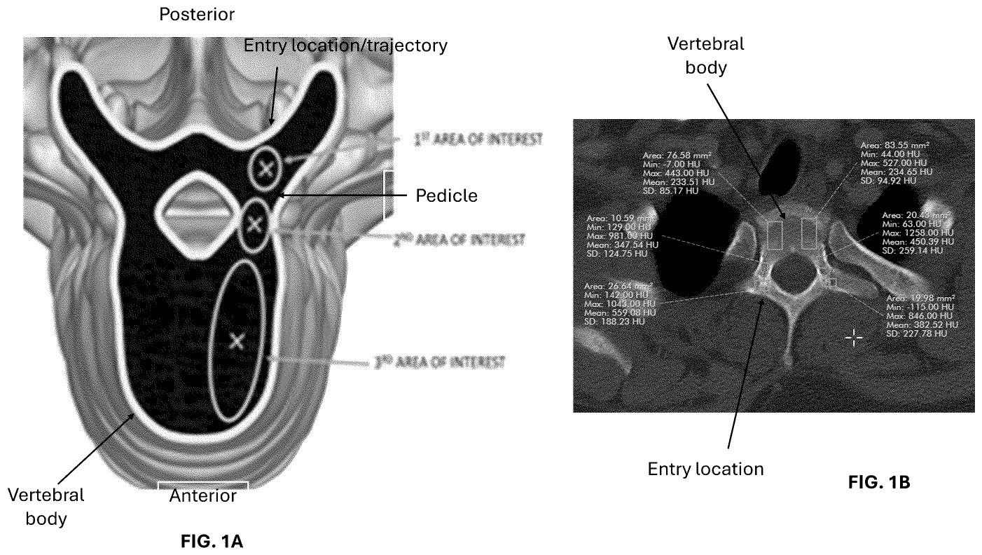

One aspect of this disclosure is related to threaded bone implants, which may be particularly suited for use as pedicles screws, although they may find applications and uses in other areas of the body. Screws herein include one or more features that provide added stability by promoting bony ingrowth and reducing the likelihood of toggle (movement caused by a force perpendicular to the screw long axis) and pullout (movement caused by a force parallel to the screw long axis) following positioning in bone. The one or more screw features are based on vertebra morphology, such that the screws may be particularly adapted for use as pedicle screws. FIGS. 1A and 1B illustrate exemplary implant corridors and generally trajectories for implantation through a pedicle and into the vertebral body. First, second and third anatomical areas or regions of interest are shown, where the areas have generally different densities and dimensions (e.g., lengths, widths). At least some of the screw features herein provide for enhanced stability by being particularly adapted for the region of interest of the bone in which they are positioned. Additionally, at least some of the screws features herein include space or volume for bony tissue to enter the screw during and after implantation, enhancing bony ingrowth (fusion) and increasing stability.

In general, the second region of interest is relatively more dense cortical bone, the third region of interest is relatively less dense cancellous bone, and the first region of interest has a density slightly less than the second region but greater than the third region. The region of the screw situated in the less dense cancellous bone receives the fewest or least loading forces, and thus need not be as strong as more proximal screw regions that are situated in the dense cortical bone and in the first region of interest. There may be more design options for the third region of the screw because it does not need to be as strong as regions of the screw to be placed within the relatively denser bone.

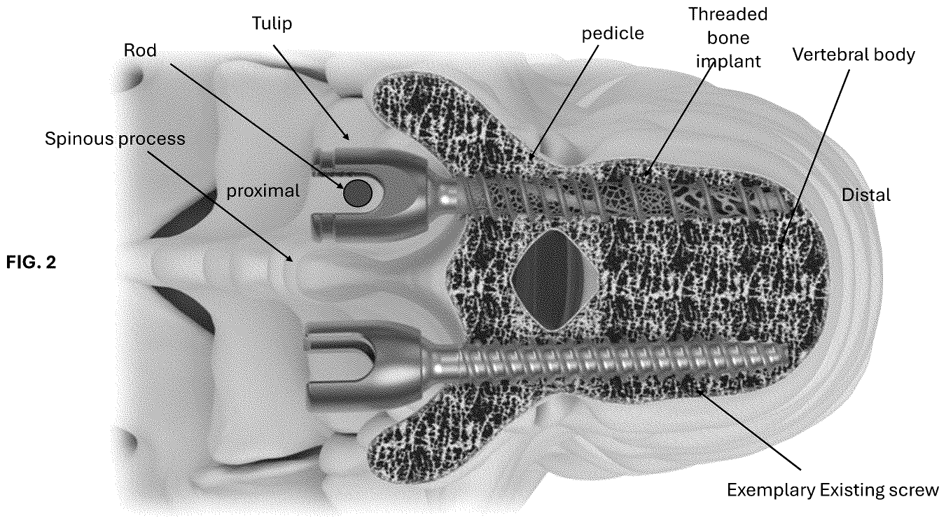

FIG. 2 illustrates an exemplary screw herein (top screw) that includes one or more screw features for added stability as a pedicle screw, compared with an exemplary existing screw (bottom screw). The corridor and trajectory shown in FIG. 2 are exemplary, as there may be some slight medial or lateral variation in trajectory/corridor (e.g., at some positive or negative angle to that which is shown in FIG. 2).

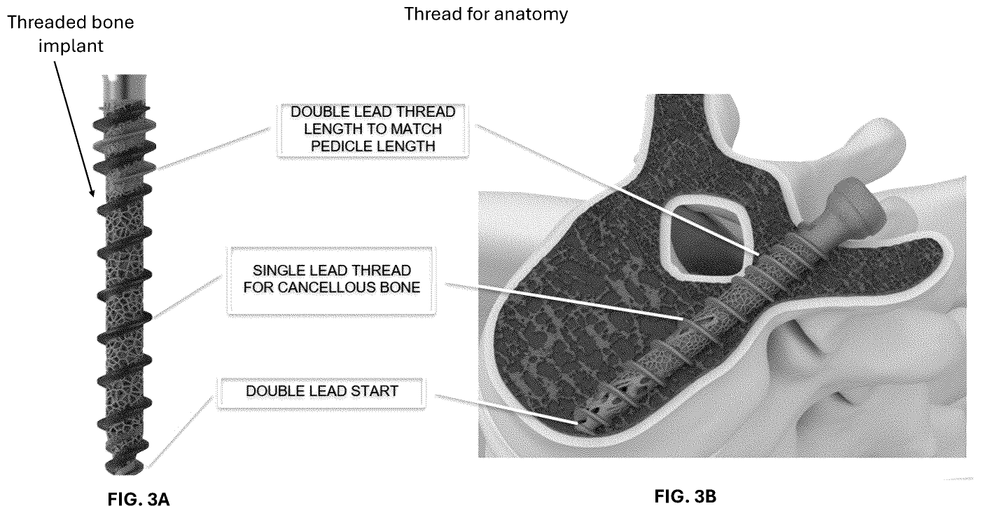

An exemplary screw feature configured to increase stability includes multi-lead thread sections (e.g., dual lead) in the second and first regions of the bone to facilitate cortical purchase. As set forth above, the screw should be strongest in the second region of interest, and optionally just as strong or close to it in the first region of interest. FIGS. 3A and 3B illustrate different screws with common features for increased stability when used as pedicle screws. As shown, the screws include a proximal region with dual-lead threads for positioning in the first and second regions, including the pedicle, at least a portion of which includes dense cortical bone. The screws include a single lead region that is positioned within the relatively less dense cancellous bone of the vertebral body. The screws also include a dual lead start distal region, with one of the distal leads reducing in height and transitioning to the minor diameter of the single lead region.

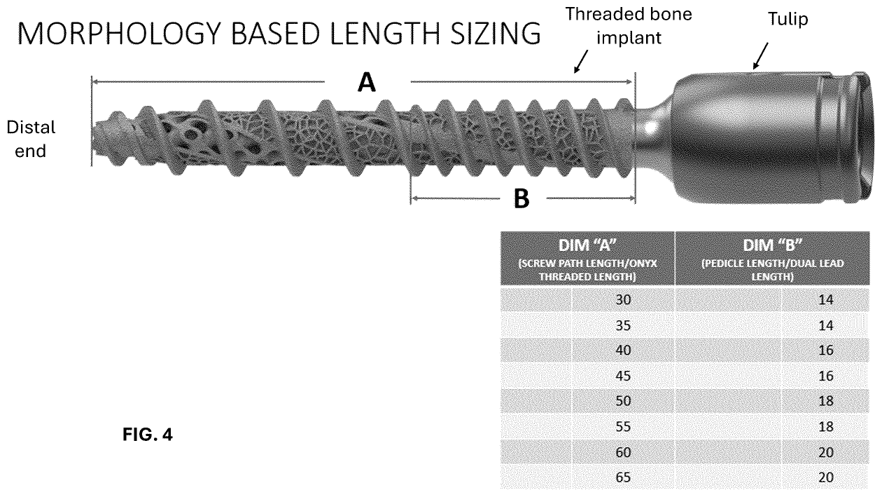

FIG. 4 illustrates an exemplary screw coupled at its proximal end to an exemplary tulip. Dimension “A” provides examples of the length of the threaded region (screw path length), for a distal end of the screw path to a proximal end of the screw path). Dimension “B” provides examples of the length of a dual thread region of the screw positioned and configured for placement at least partially in the pedicle, for cortical purchase.

Screws disclosed herein may optionally have screw path length from 20 mm to 70 mm, with examples shown in FIG. 4. Screws herein may optionally have dual lead regions from 12 mm to 25 mm, with examples shown in FIG. 4.

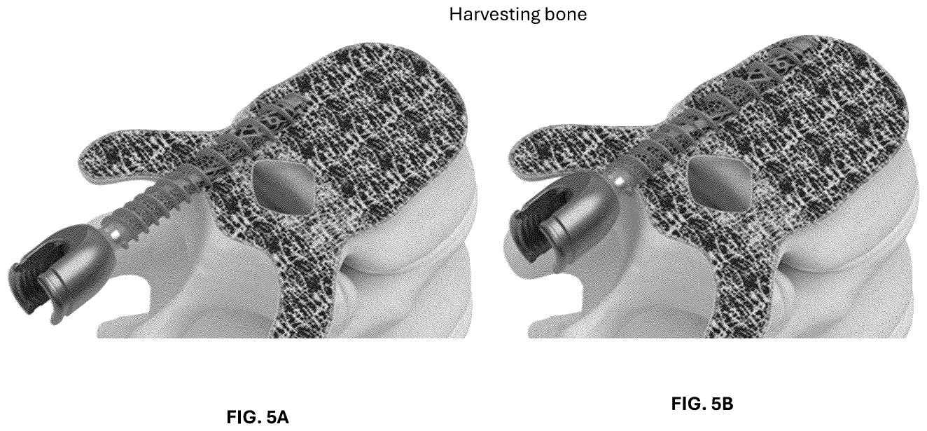

Screws herein may be adapted with one or more openings (fenestrations/pores) to facilitate self-filling harvesting of bone tissue during implantation by causing or allowing bone to enter into volume or space defined by one or more openings and optionally into an inner screw volume or cannula, thereby increasing screw stability by promoting at least one of bony on-growth, in-growth, or through-growth (all referred to herein as bony growth). The regions of the screws with one or more bone harvesting openings may be referred to herein generally as fenestration regions, flute regions, or flutes. The fenestration regions may comprise a plurality of individual fenestrations (which may also be referred to herein as pores). The fenestrations (pores) may, together, also be referred to herein as fenestration region “volume” or “fenestration volume.” Bone may enter into the fenestrations during screw insertion and pass into an inner volume or cannula, facilitating bony growth and adding screw stability. FIG. 5A represents a screw herein being rotated into bone during an implantation procedure (following an initial entry), while FIG. 5B represents the screw fully seated in place with a distal end in the vertebral body.

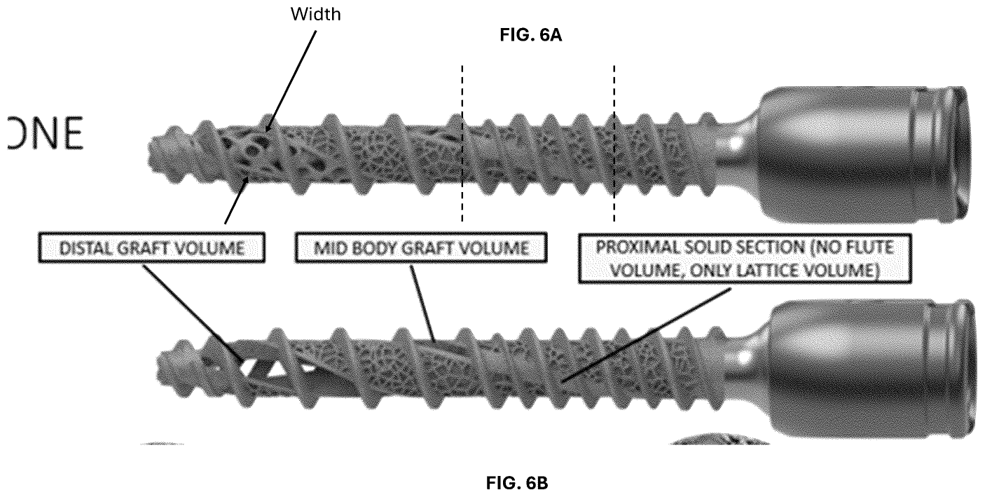

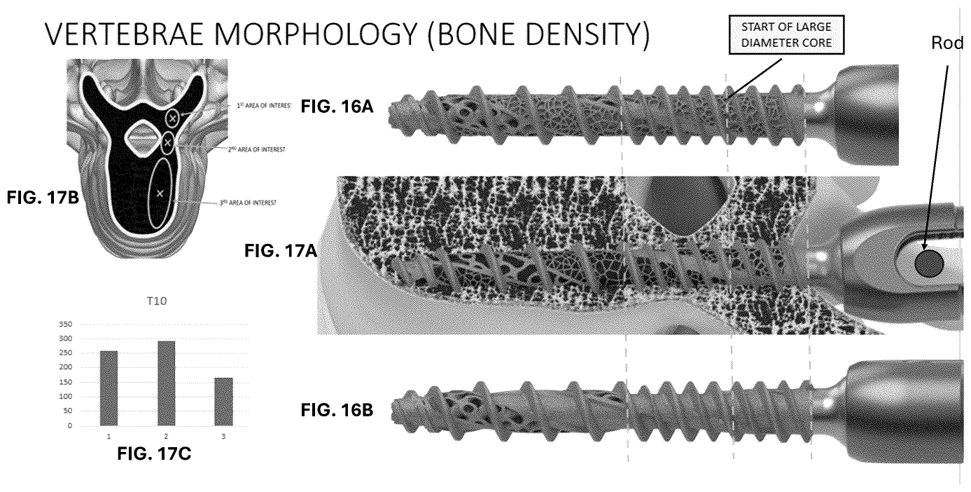

FIGS. 6A and 6B show side views of an exemplary screw configured for bone harvesting. FIG. 6A includes dashed lines which are shown below in reference to FIGS. 16A, 16B, and 17. FIG. 6B hides the particular fenestration shapes, showing generally larger fenestration regions. The fenestration region ends at the proximal end of the “third” screw section, as shown. FIG. 6B illustrates that screws herein may have fenestrations regions with varying widths along the length of the fenestration region, with the width dimension shown as the distance between the two arrows in FIG. 6A. As shown, the width is larger at the distal end of the fenestration region than at the proximal end of the fenestration region. The transition in width may be gradual (tapered), it may include a constant decrease in width, it may have one or more regions with more abrupt width changes than other regions, etc, or it may have a constant or increasing width. In this example, the fenestration region volume decreases in the proximal direction. In FIG. 6B, the location labeled mid body graft volume is a fenestration region with a smaller width than a width at the distal graft volume. In this example the fenestration region is tapered with a decreasing width in the proximal direction.

The phrase fenestration region as used herein can refer to the general portion of the screw that includes fenestrations, regardless of the particular shape of the fenestrations. Fenestrations herein are in communication with an inner volume.

Additionally, as shown in FIGS. 6A and 6B, the first and second screw regions do not include a fenestration region (free of fenestrations), but rather are solid sections, a portion of which includes a porous lattice volume, as shown. As described herein, it is generally desirable that these regions are strong, and a lack of openings/fenestration therethrough makes these regions stronger relative to the region of the screw with fenestrations. A central lumen optionally extends through the solid proximal section (the combination of first and second screw regions) and continues to the distal region.

Because the third screw region is in the less dense cancellous bone and receives fewer or less loads, it need not be as strong relative to the screw section in the denser cortical bone. This allows the third screw region to be weaker, providing the ability to include openings therethrough to provide for bone harvesting, bony ingrowth and added stability.

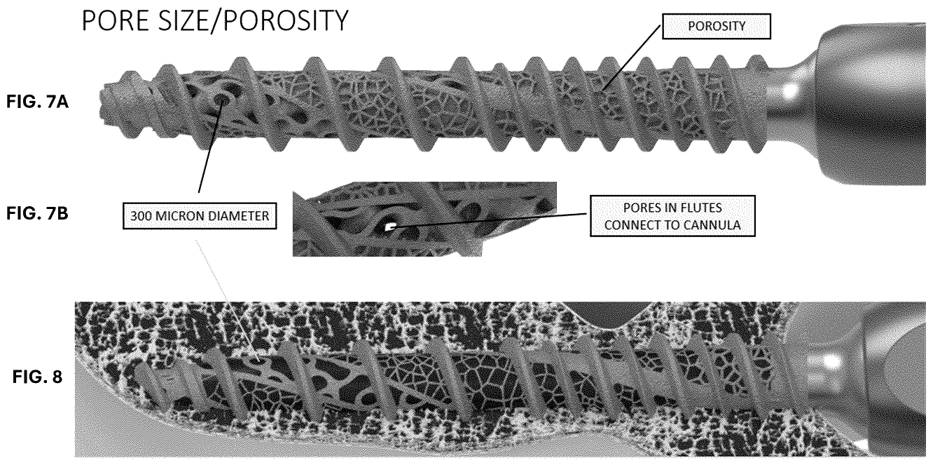

FIGS. 7A, 7B and 8 provide additional views and exemplary details of screws herein with bone harvesting features. All disclosure related to FIGS. 6A and 6B is optionally incorporated into FIGS. 7A, 7B, and 8. As shown, the third screw section includes a fenestration region with a plurality of fenestrations (which are referred to as “pores in flutes” in FIG. 7B) communication or connecting with an inner cannula/lumen/volume. As shown and as described above, the fenestration region ends at the transition to the intermediate or second screw region, wherein it/they transition to a solid support rib (upon which no lattice is built), as shown. Fenestration sizes may vary, and may vary between fenestrations within the fenestration region. A merely exemplary 300 micron diameter size is shown, but fenestrations may have other dimensions, and if the fenestration has an irregular shape, the fenestration may not have a particular size. In the proximal screw region (the first and second screw regions), the screw includes lattice sections, exemplary features of which may be found in WO2021/108590A1 and WO2021/168269A1, which are fully incorporated by reference herein in their entireties. The lattice sections provide volume for ongrowth and ingrowth. The fenestrations and lattice sections pores may both be referred to herein as “pores” or have “porosity,” while fenestrations are referred to herein as pores, or apertures, that communicate with an internal volume or space of the screw. Fenestrations herein may have generally larger pore or aperture size, but some fenestrations, such as if the fenestrations have some irregularity in their shape, may have similar sizes to the lattice pores. In the examples shown herein, at least some of, and optionally all of the fenestration “pores” are larger in size than the lattice pores.

The screws herein generally have a large volume of space, particularly compared to typical pedicle screws, for bone to enter into the screw and facilitate bone integration, fusion, and increase stability.

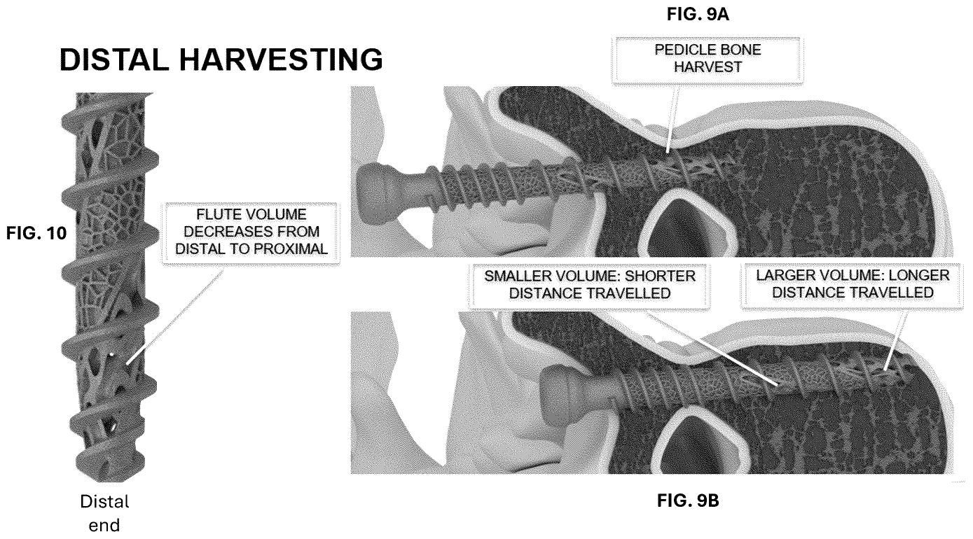

FIGS. 9A and 9B illustrate parts of an exemplary pedicle screw implantation procedure, with FIG. 9A showing a screw partially implanted and FIG. 9B showing it fully seated. During insertion, as shown in FIG. 9A, dense pedicle bone is harvested into the fenestrations openings and central cannula or lumen. FIG. 9A illustrates that a distal section of the fenestration region with relatively larger fenestrated region volume travels further through bone than fenestration regions with smaller volume (e.g., not as wide).

FIG. 10 again illustrates an exemplary distal region of any of the screws herein, showing fenestration volume decreasing from distal to proximal direction. It is understood that the screw shown in FIG. 10 is merely representative of screws herein, and not every screw includes every feature shown, and other screw features herein may be incorporated into the screw shown and/or replace screw features shown.

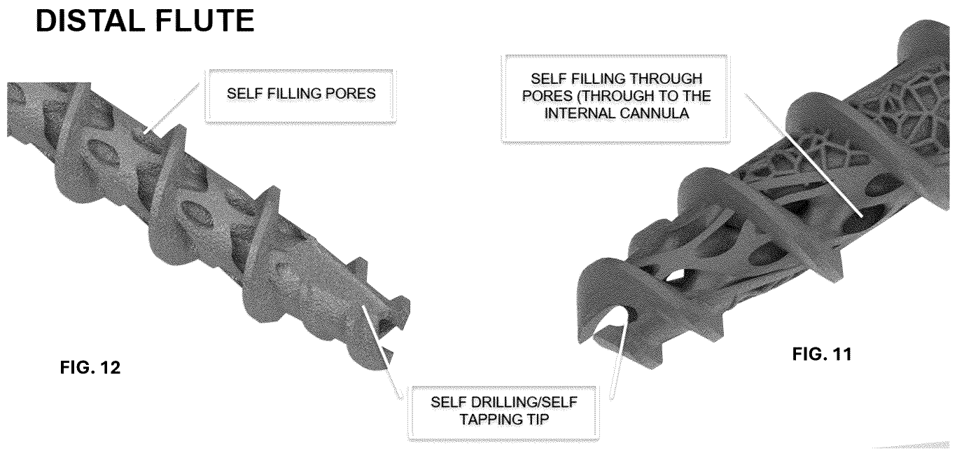

FIG. 11 shows an exemplary screw distal region (including distal end) including a self-drilling or self-tapping tip with one or more self-drilling features. FIG. 11 also illustrates an exemplary fenestration region with fenestrations, as well as lattice sections circumferentially adjacent the fenestration region (both have general helical configurations). It is understood that the screw shown in FIG. 11 is merely representative of screws herein, and not every screw includes every feature shown, and other screw features herein may be incorporated into the screw shown and/or replace screw features shown.

FIG. 12 illustrates an alternative exemplary screw with a plurality of self-filling pores and a self-drilling or self-trapping tip with one or more self-drilling features. It is understood that the screw shown in FIG. 12 is merely representative of screws herein, and not every screw includes every feature shown, and other screw features herein may be incorporated into the screw shown and/or replace screw features shown.

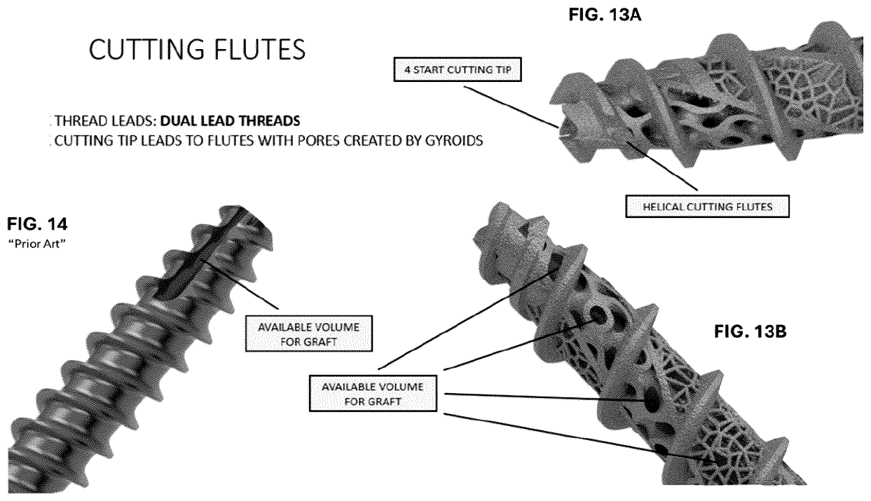

FIGS. 13A and 13B illustrate a distal region an exemplary screw herein, compared to a typical pedicle screw in FIG. 14. The screw may have a 4-start cutting tip, as shown. The screw may have a distal tip section with dual lead threads, as shown, with the minor diameter increasing from distal to proximal and one of the leads reducing in height until is transitions to the minor diameter, as shown. The screw may include helical cutting flutes, as shown. As shown, the cutting tip leads to the plurality of fenestrations, which in this example have gyroid configurations. FIG. 13B illustrates the large volume available for graft material created by the fenestrations, compared to an existing screw in FIG. 14. It is understood that the screw shown in FIGS. 13A and 13B is merely representative of screws herein, and not every screw includes every feature shown, and other screw features herein may be incorporated into the screw shown and/or replace screw features shown.

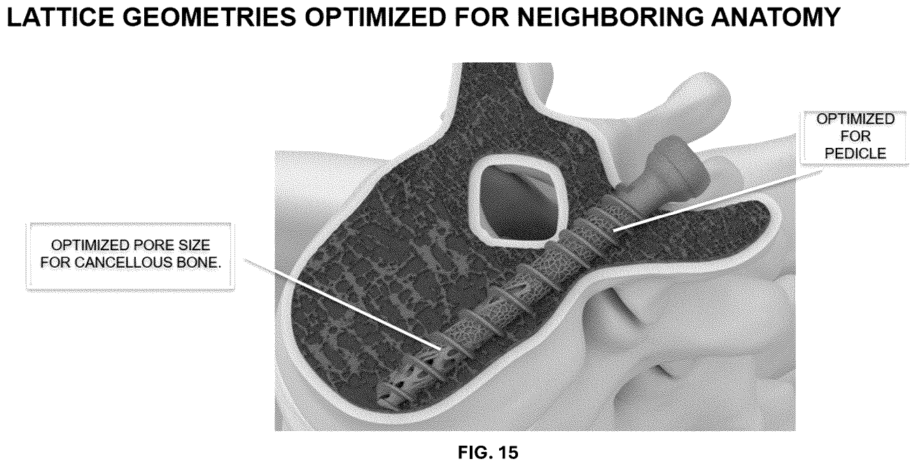

FIG. 15 illustrates that screw features herein can be particularly suited for pedicle screw placement to increase stability of the screw once implanted. As shown in FIG. 15, the screw has a distal region with a helical fenestration region with a plurality of fenestrations in communication with a central lumen, the helical fenestration region volume decreasing in the proximal direction (and tapering in this example with a decreasing width), wherein the fenestration region stops at the second region. The distal region also includes a helical lattice section. In this example, the lattice from the distal region continues to the screw region that is proximal the distal region, and in fact the lattice extends helically and completely around the proximal region, as shown in this example. In other examples the fenestration region transition to a solid region that may not have a lattice structure built up upon it. It is understood that the screw shown in FIG. 15 is merely representative of screws herein, and not every screw includes every feature shown, and other screw features herein may be incorporated into the screw shown and/or replace screw features shown.

FIGS. 16A and 16B show side views of exemplary screw, with FIG. 16B not showing the lattice sections from 16A. FIG. 16B illustrates the helical configuration of the solid regions upon which the lattice sections are formed. As shown in FIG. 16A, the distal fenestration regions transition to solid helical support rib upon which lattices are not built or constructed (in this example).

FIGS. 16A, 16B, and 17A illustrate an exemplary screw, and are shown with three common hashed lines generally separating the screw into three regions with features particularly suited for increasing the screw stability from the implantation process and/or placement adjacent particular bony tissue, examples of which are described herein. The “third” screw region (left-most) includes a dual lead distal end (with one lead transitioning in height to the minor diameter as discussed herein), fenestrations (in this example with optional gyroid shaped fenestrations), a single lead section proximal to the dual lead region, and lattice sections helically in between the larger fenestration region. The fenestrations are in communication with a central lumen that extends along the length of the screw. As shown, the fenestrations (or the fenestration region) end or stop at the proximal end of the third region (at the left dashed line). In this example the first and second screw regions are free from fenestrations but include the lattice sections, which follow a general helical configuration between the threads about at least a portion of the screw.

As is described in more detail elsewhere herein, the core diameter in this example increases abruptly in a screw proximal region (illustrated in FIG. 16A), and in this example the larger core diameter transition is designated with the dashed lines separating the first and second screw regions. By the time the first region is being rotated into bone, that boney region may have been over-prepped (preventing good thread seating), so the increase in core diameter in the first region is adapted to regain any lost bony interface due to the third and second screw regions already being rotated through it. It is understood that the screw shown in FIGS. 16A and 16B is merely representative of screws herein, and not every screw includes every feature shown, and other screw features herein may be incorporated into the screw shown and/or replace screw features shown.

FIG. 17A illustrates the screw from FIGS. 16A and 16B after positioning as a pedicle screw, extending from the cancellous bone in the vertebral body at its distal end, through the more dense cortical bone of the pedicle, through the slightly less dense bone in the first region, and extending out of the vertebra at its proximal end, which is coupled to an exemplary tulip (illustrative construct rod position shown). The three general bony regions are shown, with screw features, in this example, sized, configured and position to increase overall screw stability once positioned. FIG. 17B illustrates the three anatomical regions of interest (also shown in FIG. 1A), and FIG. 17C shows illustrative relative densities of the three regions, with the second region having the highest density and third region (cancellous bone) having the lowest density.

One of the types of screw features herein that may be sized, configured and positioned to increase screw stability include one or more aspects of thread, such as any of the number of thread leads (in one or more different section of the screw), the thread configurations, the thread height.

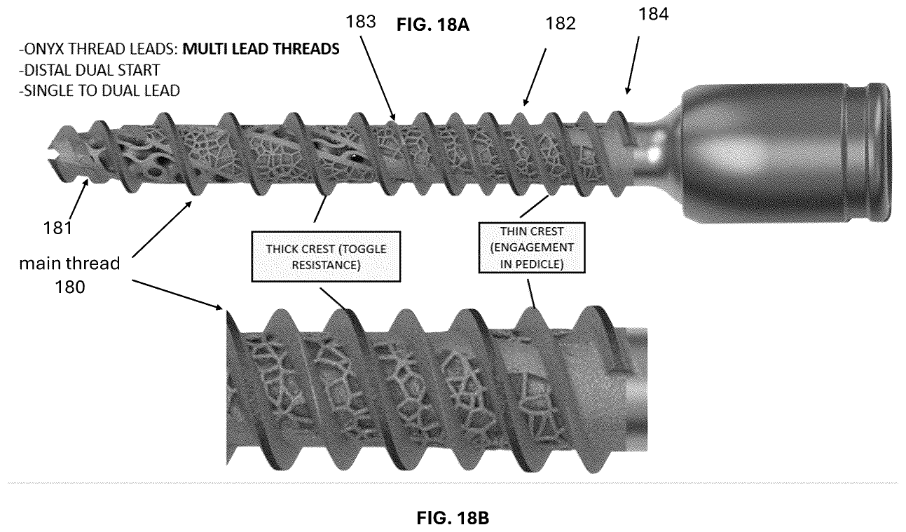

FIGS. 18A and 18B illustrate an exemplary screw adapted for increased stability, illustrating a dual-single-dual thread arrangement, which is also described elsewhere herein (shown with a tulip coupled to proximal end region). In this example, one of the threads extends from a distal thread end to a proximal thread end, as shown in blue and labeled as main thread 180. The distal region has a dual start, with second thread 181 shown in orange, which eventually transitions in height to the minor diameter as shown. A cutting tip is also shown, examples of which are shown in more detail herein. At or directly adjacent the proximal end of the fenestrated region, a proximal second thread 182 starts at 183 and continues proximally to the proximal thread end 184.

The second thread lead 181 at the distal tip helps dig into bone to get a good start. The height of lead 181 that decreases to the minor diameter and essentially goes away provides the benefit that there is no tract (or minimal tract) created in bone between the main blue thread 180, which provide for better purchase/anchoring into bone.

As is described in more detail below, all of the threads in this example have general V shaped threads (as shown), which provides better pullout performance than some other configurations. Additionally, multi-lead thread regions generally have better pullout performance. Main thread 180 has a thicker crest (e.g., square crest) than second lead 182, which can provide better toggle performance (toggle refers generally to cranial-caudal loading of the screw perpendicular to the long axis and/or movement in response to forces perpendicular to the long axis), which increases screw stability. Thicker crests have more surface area at the crest, which is more resistant to side or perpendicular forces.

Second lead 182 is provided in the proximal region, which introduces a second thread in that region (and can therefore help with pullout in the dense bone). Second lead 182 includes a thinner crest than main lead 180, as shown, which allows second lead to not take up or occupy as much space, reducing the impact on pull out performance while providing a second thread. It is understood that the screw shown in FIGS. 18A and 18B is merely representative of screws herein, and not every screw includes every feature shown, and other screw features herein may be incorporated into the screw shown and/or replace screw features shown.

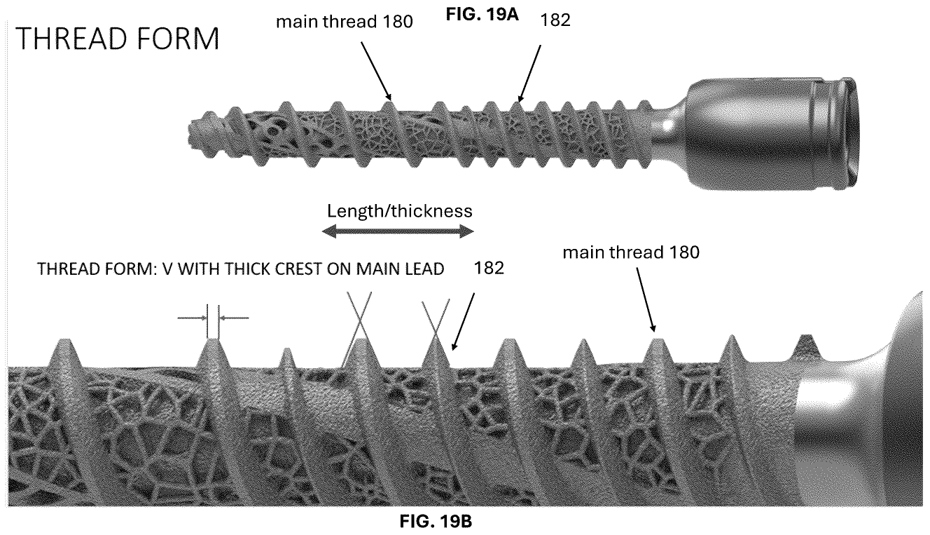

FIGS. 19A and 19B illustrate main thread lead 180 and secondary thread lead 182, showing the greater crest thickness of thread 180 compared to secondary lead 182. Crest thickness is measured in a direction along the length of screw, as shown by the double arrow line in FIG. 19B. FIG. 19B shows a portion of the screw shown in FIG. 19A, including the transition from a single lead region to a dual lead region. FIG. 19B also illustrates the proximal end of the fenestration region and optionally how it transitions to a solid screw region upon which lattice section is not present. It is understood that the screw shown in FIGS. 19A and 19B is merely representative of screws herein, and not every screw includes every feature shown, and other screw features herein may be incorporated into the screw shown and/or replace screw features shown.

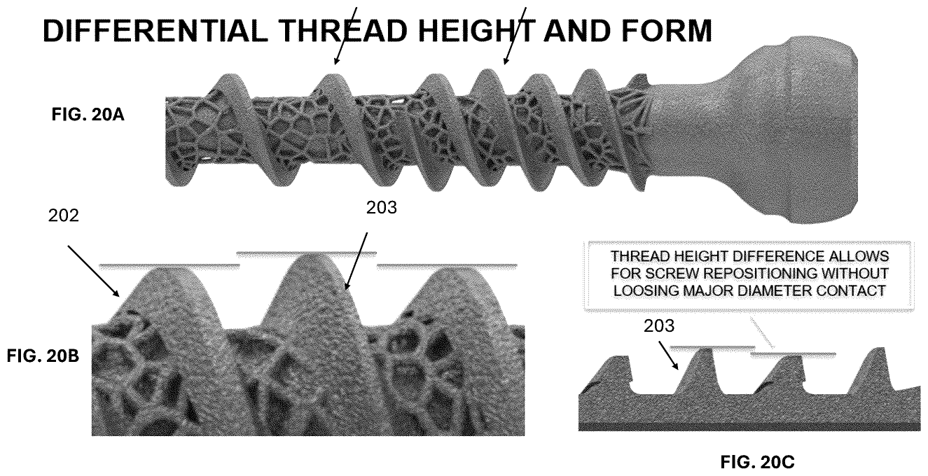

Screws herein may include a proximal dual lead section wherein the different threads have different threads heights, as illustrated in FIGS. 20A-20C. In FIGS. 20A-20C, the screw includes thread 203, which starts to create the dual lead section, which has a greater height than thread 202, which extends distally relative to thread 203 into the single lead section. This design provides thread height differential in two separate leads, which does not sacrifice toggle performance as tapered threads are prone to do. It is understood that the screw shown in FIGS. 20A, 20B, and 20C is merely representative of screws herein, and not every screw includes every feature shown, and other screw features herein may be incorporated into the screw shown and/or replace screw features shown.

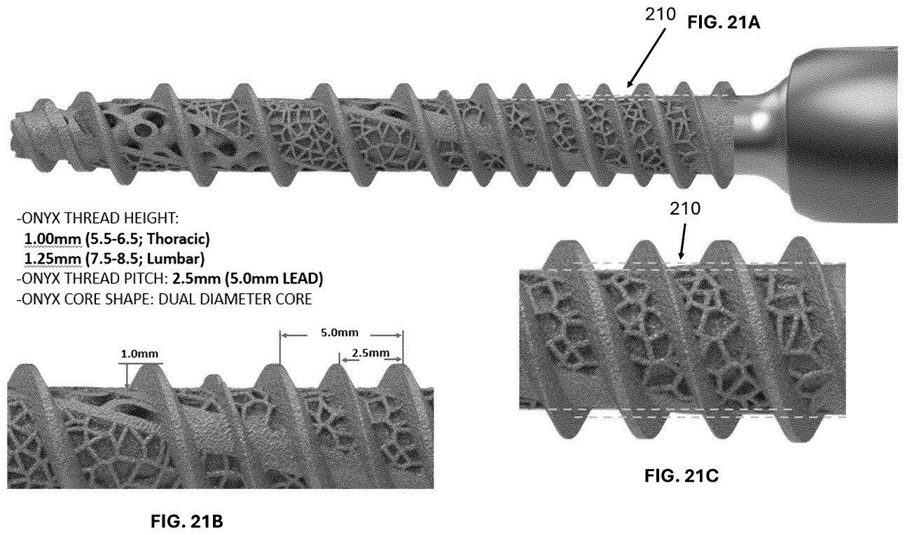

FIGS. 21A, 21B and 21C illustrate an additional example of a screw adapted to increase stability. FIG. 21B highlights a portion of the screw, illustrating exemplary dimensions. For example, the thread height for the main thread and/or the second lead may be from 1-1.5 mm, with the example shown as 1.0 mm. The thread height may also depend on the level at which the screw is inserted. For example, thread height in one or both leads may be greater in lumbar spine levels, such as 1.25 mm. In this example, lead pitch is 5.0 mm (per each lead), while thread pitch between different leads is 2.5 mm. The lead pitch and thread pitch may vary, however. In general, relatively taller threads are better than shorter threads with respect to pullout in higher density bone.

FIGS. 21a and 21C further illustrate a dual diameter aspect of screw cores herein that can help prevent pullout and provide good purchase in proximal region of bone. The first region of the bone may tend to get over-prepped, so a larger diameter proximal section starting at location 210 (shown in FIGS. 21a and 21C) can help regain any lost bony interface due to the more distal regions of the screw going through the bone first. The increase in minor diameter at location 210 is an abrupt or rapid transition, optionally being only about 2-3 mm in length between the smaller diameter section and the larger diameter section. The difference in diameters may optionally be 0.5 mm to 3 mm. The start of the larger diameter section is also shown in FIGS. 16A and 16B. The length of the “third” screw region with the larger diameter may optionally by from 4 mm to 7 mm. It is understood that the screw shown in FIGS. 21A, 21B, and 21C is merely representative of screws herein, and not every screw includes every feature shown, and other screw features herein may be incorporated into the screw shown and/or replace screw features shown.

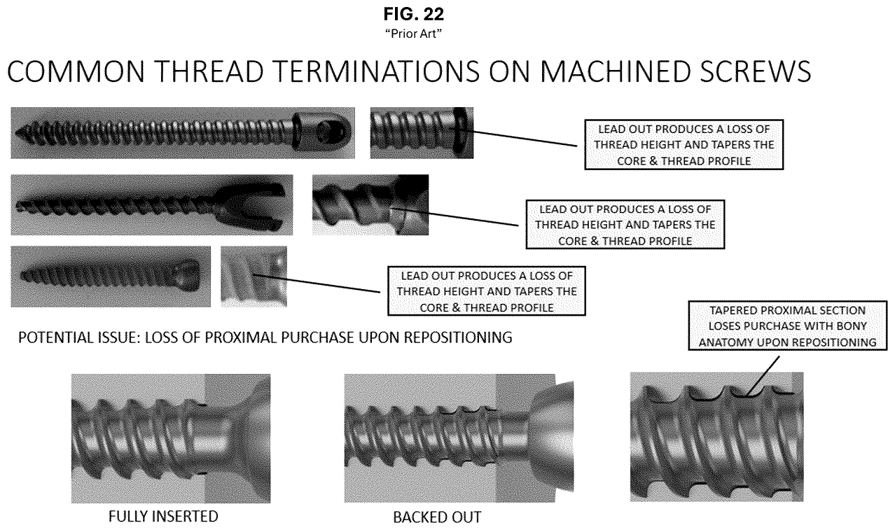

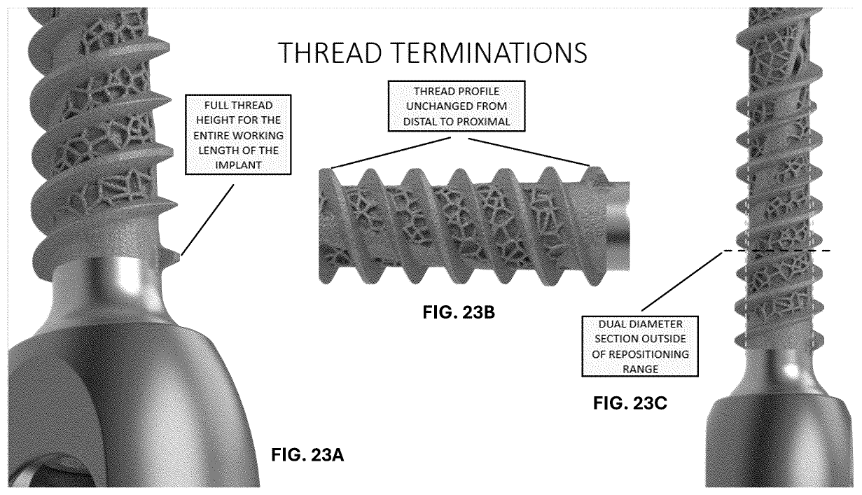

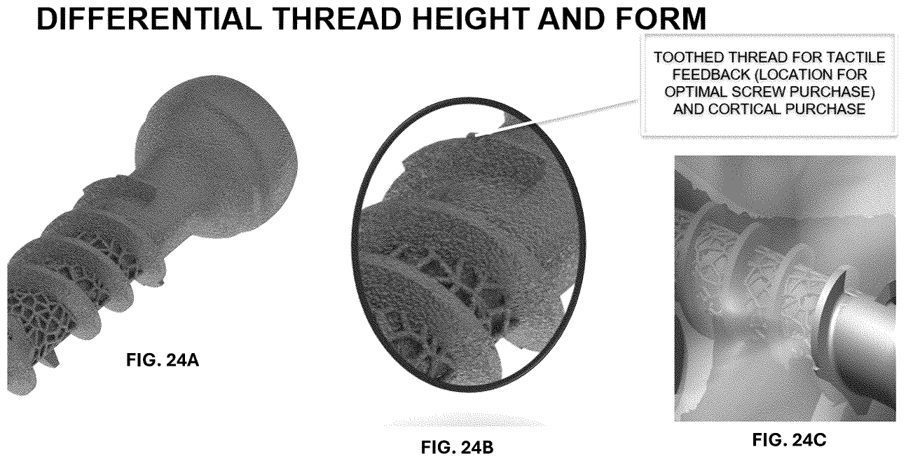

FIG. 22 illustrates some exemplary deficiencies of machined screws in the event of repositioning in bone, including a tapered core at the proximal end and a reduction in thread height at the proximal end, which occurs during machining screws based on some manufacturing techniques. Tapered cores and/or reducing in thread height can cause undesired screw/bone interface, including having a shorter proximal thread sitting in a space created by a larger height thread region, which is an undesirable interface and can reduce screw stability (e.g. causing toggle and/or pullout, which screws disclosed herein can avoid). FIGS. 23A, 23B and 23C illustrate exemplary screw features that can overcome or minimize the deficiencies of machined screws shown in FIG. 22. As shown in FIG. 23A, the thread can have the same thread height for the entire working thread length of the implant (or at least along a portion including the proximal end), including at the proximal end region, providing better purchase upon the need to reposition, unlike the tapered proximal section machined screw (including a loss of thread height) shown in FIG. 22. Additionally, as shown in FIG. 23B, the thread profile can remain unchanged all the way to the proximal end of the thread length of the screw. Additive manufacturing (e.g., 3D printing) techniques can allow these features to be manufactured into the screw and provide the added benefits. Additionally, as shown in FIG. 23C, the dual diameter section (i.e., with a larger minor diameter) remains outside of the repositioning range, and thus if the screw is repositioned, a second screw would be inserted. It is understood that the screw shown in FIGS. 23A, 23B, and 23C is merely representative of screws herein, and not every screw includes every feature shown, and other screw features herein may be incorporated into the screw shown and/or replace screw features shown.

FIGS. 24A, 24B, and 24C provide an exemplary feature that may be incorporated into any of the screws herein. One or more threads may be toothed (as shown) for tactile feedback (location for optimal screw purchase) and cortical purchase.

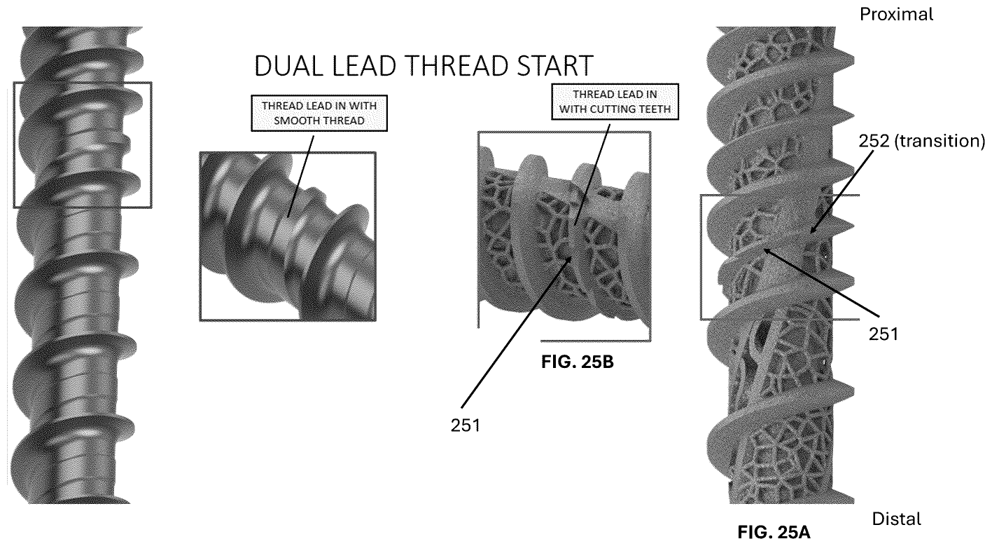

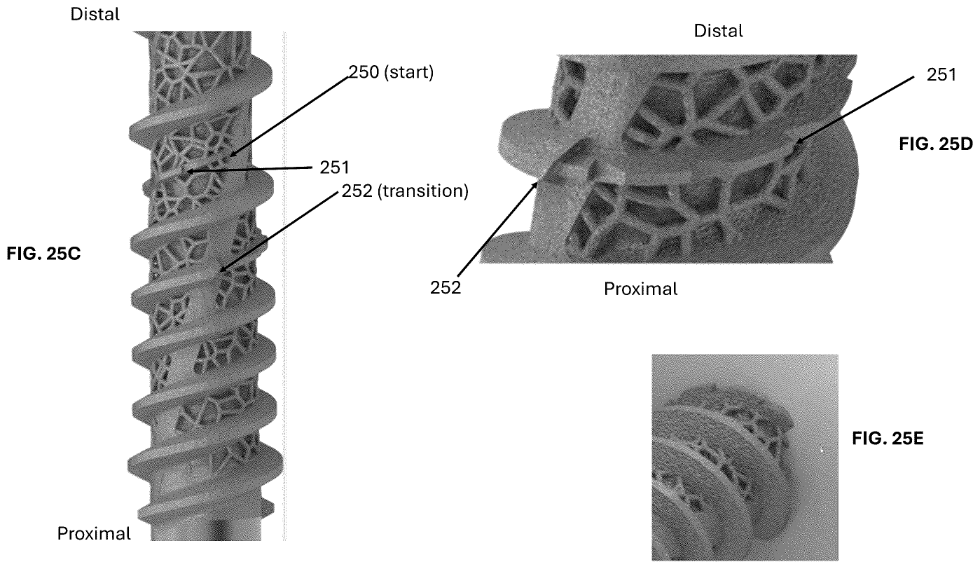

One of the screw features that may help improve stability is a feature that helps facilitate a smoother start for the second lead in the multi-lead proximal section as is advances through the high density cortical shell. The cortical shell is high density bone and preserving it can improve the pullout performance of the screw. The cutting teeth can, for example, prevent wedging into bone and avoid microfractures. An example of this screw feature herein is shown in FIGS. 4, 6A, 6B, 7A, 8, 16A, 16B, 17, 18A, 19A, 19B, 21A, 21B, 23C, 25A, 25B, 25C, 25D and 25E. As shown in FIGS. 25A-25E, the thread lead includes a plurality of cutting teeth 251 at a distal end to facilitate a smoother start (reducing the disruption to bone) and help preserve the cortical bone to improve pullout performance. This may also be referred to herein as a self-tapping feature. The lead in region has a smaller outer diameter than the thread height, and eventually transitions to the full thread height and eliminates the cutting teeth, as shown. FIGS. 25C and 25D illustrates lead in start 250 and transition 252 to full thread height and form. In the example in FIG. 25C, both thread leads are shown with the same outer crest thickness. FIG. 25E illustrates the cutting teeth penetrating into bone. The region with the cutting teeth (from the start to the transition) may optionally extend from a quarter revolution to one or more revolutions (e.g., one revolution). The cutting teeth region prepares the bone for the second lead (after the transition) so that it does not lose purchase. To the left of FIG. 25B is an illustration of a prior art screw with a smooth thread lead in for a second lead, which does not prepare the bone as does the thread lead in with cutting teeth as set forth herein. It is understood that the screw shown in FIGS. 25A-25E is merely representative of screws herein, and not every screw includes every feature shown, and other screw features herein may be incorporated into the screw shown and/or replace screw features shown.

Additionally, the height of the teeth can vary throughout the cutting teeth region.

In any of the screws herein, the serrated cutting teeth may optionally be incorporated into the start of the second lead at the distal tip, such as for lead 181 shown in FIG. 18A.

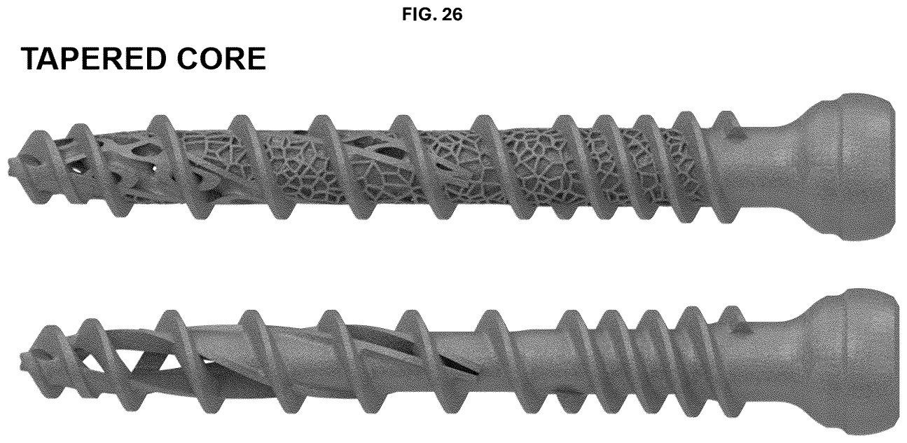

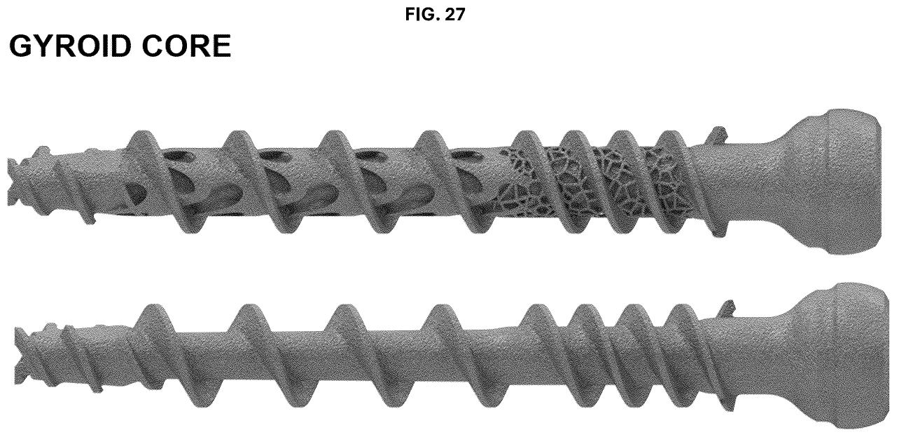

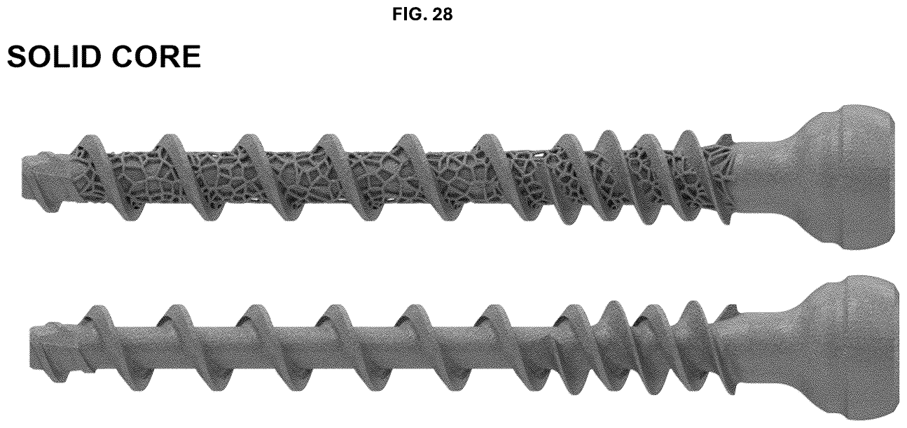

FIGS. 26, 27 and 28 illustrate exemplary core configurations, any of which may be incorporated into any of the screws herein. The tapered core design is incorporated into many of the examples herein.

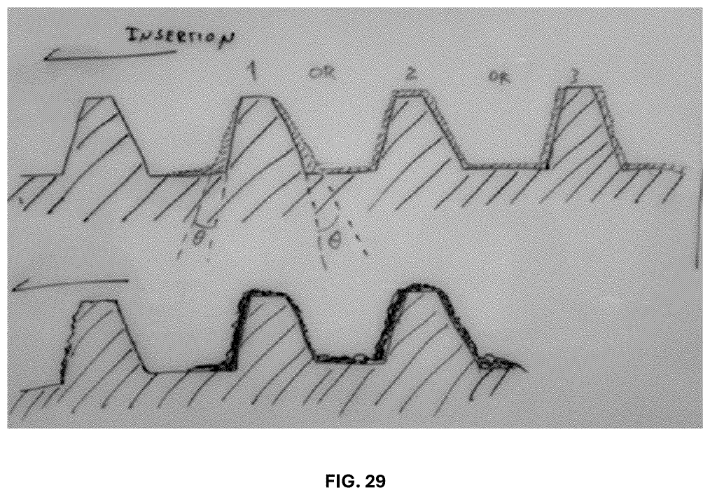

FIG. 29 illustrates additive manufacturing techniques (e.g., 3D printing) that can increase stability in screws. As shown, the technique comprises modifying the dimensions of thread surfaces (or offsetting the faces) by a very small amount along the length of at least a portion of the thread. The techniques are meant to allow threads to fill in the voids that are created in bone by thread portions that have already been threaded through the bone at that location. For example, the black regions in the bottom figure represent voids or spaces left behind by thread sections that have already been threaded through those sections (direction of insertion is the left in this example). The top figure illustrates three alternative ways to change or modify a thread dimension to increase the thread size so that it will be larger than distal thread sections and fill in the void left behind by distal thread sections. The thread dimensions could increase incremental distal to proximal, or they could change gradually distal to proximal. The red sections show additional material added to a basic thread form to increase in size in at least one dimension. In the first example, the angle may be increased to create larger thread sections. In the second example, all three surfaces of the thread may be increased to create larger thread dimensions, including an increase in major diameter as shown. In the third example, less than all surfaces are modified (i.e., no change in major diameter but outer surfaces are offset). For example only, the surfaces may be offset by a thousandth of an inch. These manufacturing techniques can be used to additively manufacture any of the screws herein.

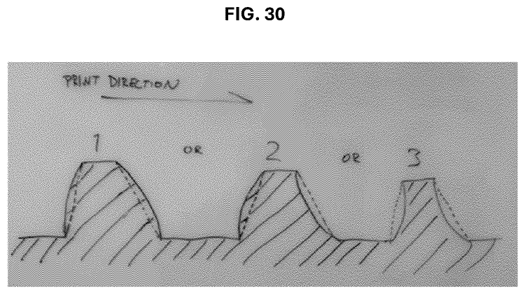

FIG. 30 illustrates exemplary additive manufacturing techniques (e.g., 3D printing) that may improve one or more aspects of any of the screws herein or other threaded implants. FIG. 30 is described in reference to a printing direction shown, bottom to top on a printing plate. The numbers 1, 2 and 3 in FIG. 30 are meant to be in the alternative (“or”), but could conceivably be incorporated into manufacture of a single screw. In FIG. 30, the dotted lines are meant to represent a 60 degree thread form.

Downward facing surfaces are prone to being printed poorly (rough) if there is a lack of support under the downward facing surface. Surfaces closer to the major diameter may even print worse than surface closer to minor diameter. Printing curved surfaces rather than straight surfaces can improve the print quality of the downward facing surface. The first example of FIG. 30 (“1”) illustrates printing a curved downward surface to improve print quality. Additionally, steeper curves print better generally, and thus it may further improve the quality by increasing the steepness of the downward surface closer to the major diameter. The print quality may not be as poor close to the minor diameter, so the curvature could be less or perhaps not even curved at all. In example “3” in FIG. 30, the technique involves undercutting the curve, so that the concave solid lines are cut. In this example, it may or may not improve print quality, but roughness created in the downward printed surface may, as the screw it rotated into place, fill out the space of the dotted line thread form, such that the thread cuts through bone close to the dotted line form. The second example “2” is meant to illustrate that it may be possible print either surface as convex or concave, and each thread is not limited only to one type of modification.

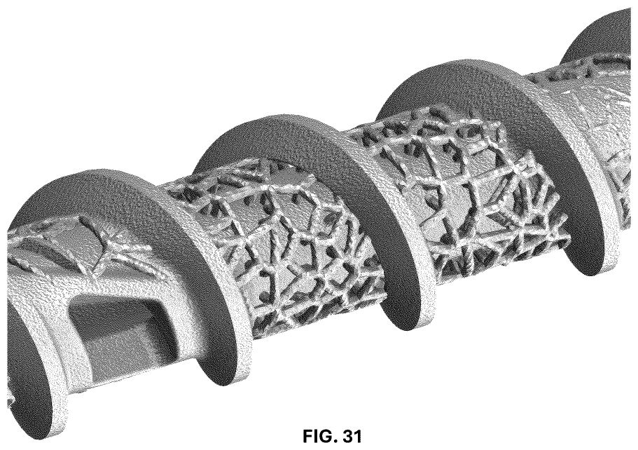

FIG. 31 illustrates a midsection of another exemplary screw. In this embodiment, rootless threads are provided on the mid body as shown. What is meant by rootless threads is threads that are connected to the minor diameter of the screw with a lattice structure as opposed to a solid structure. There is typically a stress riser at the standard thread root (the connection between the screw body and thread). This rootless thread arrangement makes the midsection more compliant and improves fatigue stress by getting rid of the stress riser. In some implementations, the threads have parallel leading and trailing faces (i.e. threads having a constant thickness from top to bottom). In other implementations, the threads have a V-shaped cross-section.

Any aspect of any example herein may be combined with any one or more aspects from any other example, and vice versa.

Any of the first, second and third screw “regions,” may also be referred to herein as “portions.”

What is claimed is:

1. A threaded bone implant (“implant”) comprising:
an elongate body comprising
a first portion, a second portion distal to the first portion, and a third portion distal to the second portion.

2. The implant of claim 1, wherein the second portion is immediately distal to the first portion, and wherein the third portion is immediately distal to the second portion.

3. The implant of claim 1, wherein the first and second portions each have greater strength than the third portion.

4. The implant of claim 1, wherein the third portion includes a fenestration region with a plurality of fenestrations therethrough in communication with an inner cannula.

5. The implant of claim 4, wherein the plurality of fenestrations have gyroid configurations.

6. The implant of claim 4, wherein the fenestration region has a width at a distal end of the fenestration region that is greater than a width at a proximal and of the fenestration region, and wherein the width tapers in a distal to proximal direction.

7. The implant of claim 4, wherein the first and second portions are solid and do not have any fenestrations that communicate with the inner cannula.

8. The implant of claim 7, wherein the first, second, and third portions include lattice structure about an inner core, and wherein at least a portion of the lattice structures have a helical configuration.

9. The implant of claim 4, wherein the fenestration region transitions to a support rib in the second portion.

10. The implant of claim 4, wherein the fenestration region has a helical configuration.

11. The implant of claim 1, wherein the first, the second, and the third portion each include at least one thread.

12. The implant of claim 11, wherein a common or main thread extends from a distal end to a proximal end of a thread length of the elongate body.

13. The implant of claim 11, wherein all threads have a general “V” shape.

14. The implant of claim 11, wherein the third region includes a distal dual lead region and a single lead region proximal to the distal dual lead region.

15. The implant of claim 14, wherein a second lead in the distal dual lead region gradually transitions in height to a minor diameter.

16. The implant of claim 11, wherein the first and second portions are dual lead sections with a first thread and a second thread.

17. The implant of claim 16 wherein the second thread starts at the transition between the second and third portion, and wherein the start of the second thread includes a plurality of cutting teeth and a height less than a height of the second thread in a region proximal to the cutting teeth.

18. The implant of claim 16, wherein the first lead has a greater thickness at a crest than the second lead, and wherein the first lead has a square crest shape.

19. The implant of claim 16, wherein the first thread has a greater thread height than the second thread.

20. The implant of claim 11, wherein a thread extends from a proximal end to a distal end of a thread length, and wherein the thread has a constant thread height along its entire length.

21. The implant of claim 1, wherein the first portion has a greater core diameter than the second portion, wherein there is a rapid transition in diameter wherein the second portion meets the first portion.

22. The implant of claim 21, wherein the rapid transition has a length from 1-5 mm in length between the smaller diameter in the second portion and the larger diameter in the first portion.

23. The implant of claim 21, wherein the difference in diameter between the second portion and first portion is from 0.5 mm to 3 mm.

24. The implant of claim 1, wherein the third portion has a length from 10 mm-40 mm.

25. The implant of claim 1, wherein the combined length of the first and second portions is from 10 mm to 25 mm.

26. The implant of claim 1, wherein the first portion has a length from 4 mm to 7 mm.

27. The implant of claim 1, wherein the third portion is longer than the first and second sections combined.

28. The implant of claim 1, wherein a distal tip region includes a plurality of cutting surface.

29. The implant of claim 1, wherein the second portion is sized to be placed within a pedicle, and the third portion is sized to be placed in a vertebral body.

30. The implant of claim 1, further comprising a thread with a thread height that is uniform or constant along an entire length of the thread.
